(19) United States
(12) Patent Application Publication
(10) Pub. No.: US 2014/0255251 A1
(43) Pub. Date: Sep. 11, 2014

(54) PLASMA STERILIZATION APPARATUS AND PLASMA STERILIZATION METHOD

(71) Applicant: Hanshin Medical Co., Ltd., Bupyeong-gu (KR)

(72) Inventor: Jung Yeul Kim, Bupyeong-gu (KR)

(73) Assignee: HANSHIN MEDICAL CO., LTD., Bupyeong-gu (KR)

(21) Appl. No.: 14/172,852

(22) Filed: Feb. 4, 2014

(30) Foreign Application Priority Data
Mar. 7, 2013 (KR) 10-2013-0024651

(51) Int. Cl. A61L 2/14

(52) U.S. Cl. CPC A61L 2/14
USPC 422/29; 422/292; 422/298

(57) ABSTRACT

Disclosed is a plasma sterilization apparatus including a chamber including a sterilization target, a vaporizer heating air, a vacuum unit discharging moisture and cold air from the chamber to the outside, an air supply unit injecting heated air into the chamber, and a control unit controlling the air supply unit to inject air heated by the vaporizer into the chamber, controlling the vacuum unit to discharge air to the outside until an internal pressure of the chamber becomes a firstly set pressure, and controlling the air supply unit to inject heated air into the chamber until the internal pressure of the chamber becomes a secondly set pressure.

### RELATED APPLICATION

This application claims priority under 35 U.S.C. §119 to Korean Patent Application Number 10-2013-0024651 filed on Mar. 7, 2013. The entire contents of the foregoing application are explicitly incorporated herein by reference.

### BACKGROUND OF THE INVENTION

1. Field of the Invention

The present invention relates to a plasma sterilization apparatus and a plasma sterilization method including a drying function of a sterilization target by preheating.

2. Discussion of the Related Art

When heat is continuously applied to a substance in a gas state to increase a temperature, an aggregate of particles formed of ions and free electrons is formed. The aggregate is called ‘a fourth substance state’ in addition to solid, liquid, and gas as three types of a substance, and a substance in this state is called plasma.

Plasma refers to ionized gas satisfying Debye shielding in physical and chemical fields. Plasma is considered as another state in addition to gas, liquid, and solid states as basic three states of the substance. Plasma has high electric conductivity and very high reactivity to an electromagnetic field due to freely moving electric charges.

Since properties of plasma are very different from those of general gas, plasma is called a fourth state of the substance. Plasma was first confirmed in a discharge tube by William Crookes in 1879.

Plasma is an agglomerate of particles having electric charges having electric conductivity and aggregatively reacts with an external electromagnetic field.

In general, plasma has an agglomerate or ion beam type such as neutral gas.

Sterilization is a process of effectively removing metastatic germs such as molds, bacteria, viruses, and sporic germs from surfaces, devices, foods, drugs, and culture media. Examples of a sterilization method include Dry heat, Chemicals, Irradiation, and High pressure steam. Particularly, a plasma sterilization method is known as an up-to-date technology to a current sterilization method.

Further, a plasma sterilizer uses hydrogen peroxide and a plasma technology. Hydrogen peroxide used as a sterilant has strong acidity, and the strong acidity helps the sterilant to destroy various pathogenic bacteria. In addition, the plasma sterilizer is used to sterilize precision medical appliances sensitive to a high temperature and a high pressure and humidity, such as hard or soft endoscopes.

However, there is a problem in that when sterilization starts in a state where a sterilization target is cold or moisture is included, a sterilization effect is reduced. Accordingly, before sterilization is performed, it is required that moisture included in the sterilization target is removed and the sterilization target is pre-heated.

### SUMMARY

The present invention has been made in an effort to provide a plasma sterilization apparatus and a plasma sterilization method, in which moisture of a sterilization target is removed by a pre-heating process of the sterilization target and a sterilization effect to the sterilization target received in a chamber is improved.

An exemplary embodiment of the present invention provides a plasma sterilization apparatus including a chamber including a sterilization target. A vaporizer heats air. A vacuum unit discharges moisture and cold air from the chamber to the outside. An air supply unit injects heated air into the chamber. A control unit controls the air supply unit to inject air heated by the vaporizer into the chamber, controls the vacuum unit to discharge air to the outside until an internal pressure of the chamber becomes a firstly set pressure, and controls the air supply unit to inject heated air into the chamber until the internal pressure of the chamber becomes a secondly set pressure.

In the plasma sterilization apparatus according to the exemplary embodiment of the present invention, the firstly set pressure may have a value that is higher than 300 Torr and lower than an atmospheric pressure. The secondly set pressure may have a value that is higher than the atmospheric pressure and lower than 900 Torr.

In the plasma sterilization apparatus according to the exemplary embodiment of the present invention, the air supply unit may include an air supply pump injecting air heated by the vaporizer into the chamber. An injection valve may be provided between the chamber and the vaporizer and opened and closed to adjust an injection flow of air heated by the vaporizer into the chamber.

Further, the control unit may operate the air supply pump to inject air heated by the vaporizer into the chamber and open the injection valve to inject air heated by the vaporizer into the chamber.

In the plasma sterilization apparatus according to the exemplary embodiment of the present invention, the air supply unit may include an air supply pump absorbing air passing through a HEPA filter from the outside and supplying filtered air to the vaporizer. A first air supply valve may be provided between the vaporizer and the air supply pump and opened and closed to adjust a supply flow of filtered air to the vaporizer.

Further, the control unit may operate the air supply pump to absorb air passing through the HEPA filter from the outside and supply filtered air to the vaporizer, and open the first air supply valve to supply filtered air to the vaporizer.

The plasma sterilization apparatus according to the exemplary embodiment of the present invention may further include a sterilant supply unit supplying a sterilant to the chamber, and a plasma generation unit generating plasma in the chamber.

Another exemplary embodiment of the present invention provides a plasma sterilization method of a plasma sterilization apparatus. The method includes heating air, exhausting air from a chamber to the outside until an internal pressure of the chamber becomes a firstly set pressure to perform a pressure reduction, and injecting heated air into the chamber until the internal pressure of the chamber becomes a secondly set pressure to perform a pressure increase.

In the plasma sterilization method according to the exemplary embodiment of the present invention, the firstly set pressure may have a value that is higher than 300 Torr and lower than the atmospheric pressure. The secondly set pressure may have a value that is higher than the atmospheric pressure and lower than 900 Torr.

In the plasma sterilization method according to the exemplary embodiment of the present invention, the exhausting of air and the injecting of heated air may be repeatedly performed.

The plasma sterilization method according to the exemplary embodiment of the present invention may further include after the injecting of heated air, reducing the pressure of the chamber to a set level, supplying the sterilant into the chamber, diffusing the sterilant for a set time, additionally reducing the pressure of the chamber, and generating plasma to perform sterilization.

As described above, it is possible to improve a sterilization effect by the plasma sterilization apparatus and the plasma sterilization method according to the present invention.

### BRIEF DESCRIPTION OF THE DRAWINGS

FIG. 1 is a view showing a plasma sterilization apparatus usable in a plasma sterilization method according to an exemplary embodiment of the present invention.

FIG. 2 is a graph showing an internal temperature and an internal pressure of a chamber according to the plasma sterilization method according to the exemplary embodiment of the present invention.

### DETAILED DESCRIPTION

Hereinafter, a plasma sterilization method according to an exemplary embodiment of the present invention will be described in detail below with reference to the accompanying drawings.

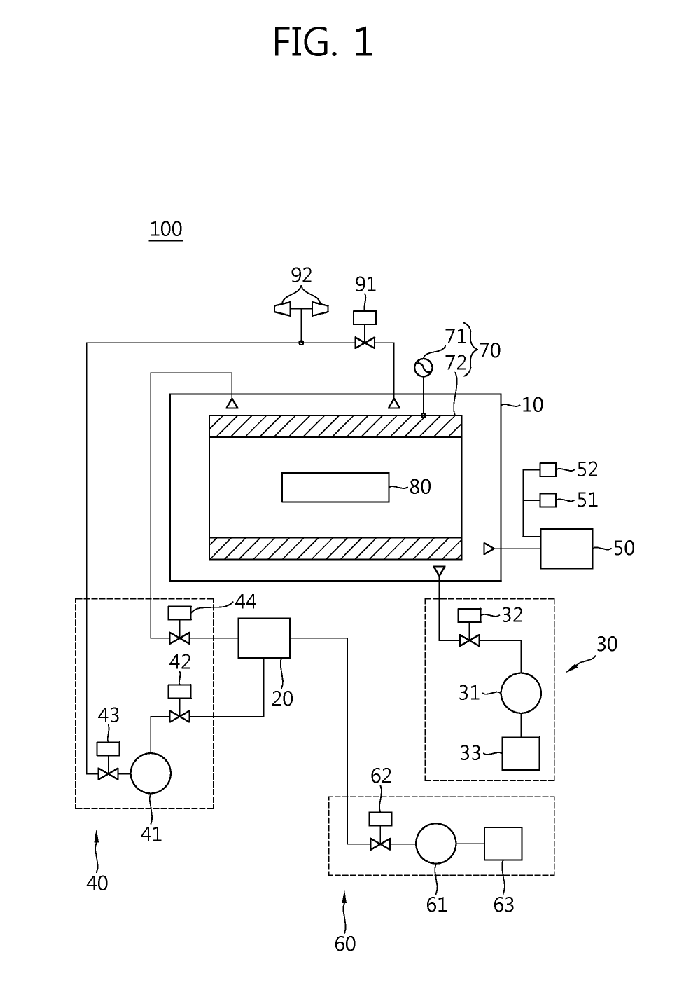

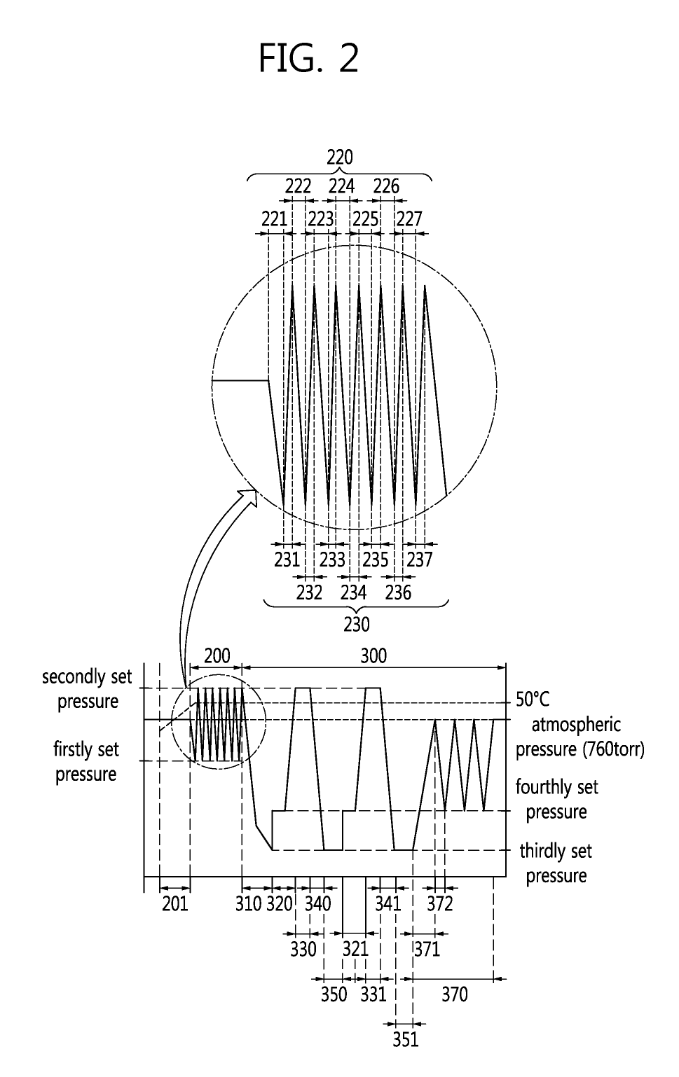

FIG. 1 is a view showing a plasma sterilization apparatus usable in a plasma sterilization method according to an exemplary embodiment of the present invention. FIG. 2 is a graph showing an internal temperature and an internal pressure of a chamber according to the plasma sterilization method according to the exemplary embodiment of the present invention.

First, a plasma sterilization apparatus 100 usable in the plasma sterilization method according to the exemplary embodiment will be described with reference to FIG. 1. Next, the plasma sterilization method according to the exemplary embodiment will be described with reference to FIG. 2.

Referring to FIG. 1, the plasma sterilization apparatus 100 usable in the present exemplary embodiment may include a chamber 10, a vaporizer 20, a vacuum unit 30, an air supply unit 40, a control unit 50, a sterilant supply unit 60, a plasma generation unit 70, an air supply valve for ventilation 91, and a HEPA filter 92.

The chamber 10 may have a space receiving a sterilization target 80 therein. In the chamber 10, heating, air absorption, airtight closing, and air exhausting may be performed to maintain set temperatures and pressures.

The vacuum unit 30 discharges moisture and air from the chamber 10 to the outside. For example, the vacuum unit 30 includes a vacuum pump 31 exhausting air from the chamber 10. The vacuum unit 30 may include an exhaust valve 32 opened and closed to control an air flow discharged from the chamber 10 to the outside. The vacuum unit 30 may further include an exhaust filter 33 filtering air exhausted from the chamber 10.

Needless to say, various types of pumps and valves may be used as the vacuum pump 31 and the exhaust valve 32. For example, a solenoid valve may be used as the exhaust valve 32. In addition, a filter using various catalysts may be used as the exhaust filter 33 in consideration of a sterilant.

For example, when a hydrogen peroxide (H₂O₂) solution is used as the sterilant, all catalysts relating to decomposition of hydrogen peroxides in addition to manganese dioxides may be used in the exhaust filter 33. The exhaust filter 33 may decompose hydrogen peroxide gas remaining after a reaction in the chamber 10 during a sterilization process into gas harmless to humans and the environment.

The vaporizer 20 heats injected external air. Further, the vaporizer 20 vaporizes the sterilant by heating before the sterilant is supplied into the chamber 10.

The HEPA filter 92 comes into direct contact with external air, and may filter a fine impurity included in external air.

The air supply unit 40 is constituted to inject air heated by the vaporizer 20 into the chamber 10. The air supply unit 40 may include an air supply pump 41, a first air supply valve 42, a second air supply valve 43, and an injection valve 44.

The air supply pump 41 connected to the HEPA filter 92 may circulate air. For example, the air supply pump 41 may provide force to absorb air from the outside and inject filtered air into the vaporizer 20. Further, the air supply pump 41 may provide force to inject air heated by the vaporizer 20 into the chamber 10.

The vaporizer 20 is provided between the air supply pump 41 connected to the HEPA filter 92 and the chamber 10. In addition, the second air supply valve 43 is provided between the HEPA filter 92 and the air supply pump 41. The first air supply valve 42 is provided between the air supply pump 41 and the vaporizer 20. The injection valve 44 is provided between the vaporizer 20 and the chamber 10.

The second air supply valve 43 may be opened and closed so that air is absorbed through the HEPA filter which is in contact with the outside. The first air supply valve 42 may be opened and closed to adjust a supply flow of filtered air to the vaporizer 20. The injection valve 44 may be opened and closed to adjust an injection flow of air heated by the vaporizer 20 into the chamber 10.

The air supply pump 41, the first air supply valve 42, the second air supply valve 43, and the injection valve 44 may use various types of pumps and valves.

The sterilant supply unit 60 may function to supply the sterilant into the chamber 10. The sterilant supply unit 60 may include a sterilant supply pump 61, a sterilant supply valve 62, and a sterilant supply tank 63.

The plasma generation unit 70 may include a power supply source 71 supplying a power for forming plasma, and an electrode 72 receiving power from the power supply source 71 to form plasma.

Various types of power supply sources and electrodes may be used as the power supply source 71 and the electrode 72. For example, the power supply source 71 may be the power supply source 71 providing a high frequency voltage. In addition, when the chamber 10 has a shape of a cylinder that is opened at a side thereof, the electrode 72 may have a cylinder shape corresponding to an internal shape of the chamber 10.

The air supply valve for ventilation 91 may be provided between the chamber 10 and the HEPA filter 92. The air supply valve for ventilation 91 may be opened and closed to adjust a flow of air flowing into the chamber 10 or exhausted. For example, the air supply valve for ventilation 91 may be closed to block the chamber 10 from the outside. Further, the air supply valve for ventilation 91 may be opened to ventilate the chamber 10 and adjust the internal pressure of the chamber 10 to an atmospheric pressure.

The control unit 50 controls an operation of the plasma sterilization apparatus 100 sterilizing the sterilization target 80.

The control unit 50 may control the vacuum unit 30 to discharge air to the outside until the internal pressure of the chamber 10 becomes a firstly set pressure that is higher than 300 Torr and lower than the atmospheric pressure. For example, the control unit 50 may open the exhaust valve 32 and operate the vacuum pump 31 until the internal pressure becomes the firstly set pressure.

The control unit 50 may control the air supply unit 40 to absorb air passing through the HEPA filter 92 from the outside and supply filtered air to the vaporizer 20. For example, the control unit 50 may perform controlling to inject air into the vaporizer 20 by operating the air supply pump 41 and opening the first air supply valve 42 and the second air supply valve 43. The control unit 50 may control the vaporizer 20 to heat supplied air by the vaporizer 20.

The control unit 50 may control the air supply unit 40 to inject air heated by the vaporizer 20 into the chamber 10 until the internal pressure of the chamber 10 becomes a secondly set pressure having a value that is higher than the atmospheric pressure and lower than 900 Torr. For example, the control unit 50 may open the injection valve 44, the first air supply valve 42, and the second air supply valve 43, and operate the air supply pump 41 until the internal pressure becomes the secondly set pressure.

The control unit 50 may control the sterilant supply unit 60 to supply the sterilant to the chamber 10. For example, the control unit 50 may open the sterilant supply valve 62 and operate the sterilant supply pump 61 to extract the sterilant received in the sterilant supply tank 63 in a set amount.

The sterilant may be first injected into the vaporizer 20 before supplied to the chamber 10. The control unit 50 may control the temperature of the vaporizer 20 to heat the supplied sterilant in a liquid state to vaporize the sterilant. Further, the control unit 50 may control the sterilant supply unit 60 and the air supply unit 40 to inject the sterilant vaporized by the vaporizer 20 into the chamber 10.

The control unit 50 may control the plasma generation unit 70 to generate plasma in the chamber 10. For example, the control unit 50 may control the plasma generation unit 70 to generate power from the power supply source 71 and transfer generated power to the electrode 72. The electrode 72 to which power is transferred may generate plasma.

The control unit 50 may control to open the air supply valve for ventilation 91 to ventilate the chamber 10. In this case, since the chamber 10 comes into direct contact with the outside while the air supply valve for ventilation 91 is opened, the internal pressure of the chamber 10 may become the atmospheric pressure.

In addition, the plasma sterilization apparatus 100 may further include a pressure sensor 51 detecting the internal pressure of the chamber 10, and a temperature sensor 52 detecting the internal temperature of the chamber 10.

The plasma sterilization apparatus 100 may further include various sensors detecting various process conditions, and a circuit breaker (not shown) automatically cutting off a power source when an electric short circuit or overheating occurs.

The plasma sterilization apparatus 100 may further include a heater (not shown) adjusting the internal temperature of the chamber 10.

The drawings and the description exemplify the plasma sterilization apparatus 100 usable in the present exemplary embodiment, but the present invention is not limited thereto. Accordingly, the sterilant may be supplied in a desired amount by other various types and/or structures. Needless to say, the plasma sterilization apparatus 100 adjusting the internal pressure and the internal temperature of the chamber 10 may be applied.

FIG. 2 is a graph showing the internal temperature and the internal pressure of the chamber according to the plasma sterilization method according to the exemplary embodiment of the present invention.

Referring to FIGS. 1 and 2, the plasma sterilization method according to the present exemplary embodiment may include a heating step 201, a pre-heating step 200, and a sterilization step 300.

In the heating step 201, a chamber jacket (not shown) and the vaporizer 20 are heated. For example, the control unit 50 may control a heater temperature of the vaporizer 20 to perform heating to a set temperature. Further, a heater of the chamber jacket may be controlled to maintain a sterilization temperature at 40 to 60° C. during the sterilization step to be performed in the chamber 10.

The plasma sterilization method may further include, after the heating step 201 is finished, disposing the sterilization target 80 such as medical or surgical devices to be sterilized in the chamber 10 and closing a door of the chamber 10 to seal an inside of the chamber 10. In this case, the exhaust valve 32, the first air supply valve 42, the second air supply valve 43, the injection valve 44, the sterilant supply valve 62, and the air supply valve for ventilation 91 are all in a closed state.

The pre-heating step 200 may include a pressure reducing step 220 of discharging air from the chamber 10 to the outside until the internal pressure of the chamber 10 becomes the firstly set pressure, and a pressure increasing step 230 of injecting heated air into the chamber 10 until the internal pressure of the chamber 10 becomes the secondly set pressure. The present invention is not limited thereto, and, of course, various modifications such as addition of a separate step may be conducted.

First, the pre-heating step 200 will be described below in more detail.

In the pressure reducing step 220, air is exhausted from the chamber 10 to the outside. For example, the control unit 50 may close the injection valve 44, open the exhaust valve 32, and operate the vacuum pump 31 to exhaust air from the chamber 10 to the outside. In this case, the control unit 50 may control the vacuum unit 30 to reduce the internal pressure of the chamber 10 to the firstly set pressure that is higher than 300 Torr and lower than atmospheric pressure.

Cold air and moisture in the chamber 10 and the sterilization target 80 may be removed by reducing the pressure.

In the pressure increasing step 230, heated air is injected into the chamber 10. For example, when the control unit 50 opens the second air supply valve 43 and the first air supply valve 42 and operates the air supply pump 41, external air filtered by the HEPA filter 92 is supplied to the vaporizer 20. The control unit 50 may control the injection valve 44 to open the injection valve 44 to inject air heated by the vaporizer 20 into the chamber 10.

In this case, the control unit 50 may control the air supply unit 40 to increase the internal pressure of the chamber 10 to the secondly set pressure that is higher than the atmospheric pressure and lower than 900 Torr.

When heated air is injected until the internal pressure becomes the secondly set pressure, heat may be sufficiently transferred to the inside of the sterilization target 80 regardless of the size or the shape of the sterilization target 80. Accordingly, moisture included in the sterilization target 80 as well as moisture on an external surface of the sterilization target 80 may be vaporized into the air in the chamber 10. Particularly, even when the sterilization target 80 has a narrow and long inner cavity such as a lumen, heat may be effectively transferred to the inside of the inner cavity.

In the drawings and the detailed description, the pressure reducing step 220 and the pressure increasing step 230 are repeatedly performed seven times, but the present invention is not limited thereto. Needless to say, the number of repetition may be adjusted in consideration of a type and a shape of the sterilization target 80 and a process condition of the pre-heating step 200.

For example, the pressure reducing step of discharging air from the chamber 10 to the outside until the internal pressure of the chamber 10 becomes the firstly set pressure that is higher than 300 Torr and lower than the atmospheric pressure, and the pressure increasing step of injecting pre-heated air into the chamber 10 until the internal pressure of the chamber 10 becomes the secondly set pressure that is higher than the atmospheric pressure and lower than 900 Torr may be repeatedly performed 2 to 20 times.

In the plasma sterilization method, the sterilization step 300 may be performed after the pre-heating step 200 of removing and drying cold air and humidity in the chamber 10 and the sterilization target 80. In the sterilization step 300, after the sterilant is injected into the chamber 10 and diffused, plasma is generated.

The sterilization step 300 will be described below in more detail.

The sterilization step 300 may include a step of reducing the pressure of the chamber to a vacuum 310, sterilant injection steps 320 and 321, sterilant diffusion steps 330 and 331, sterilant discharging steps 340 and 341, plasma generation steps 350 and 351, and a remaining gas removal step 370.

In the step of reducing the pressure of the chamber to the vacuum 310, the internal pressure of the chamber 10 is reduced to a thirdly set pressure (for example, vacuum pressure). For example, the control unit 50 may open the exhaust valve 32 and operate the vacuum pump 31 to reduce the internal pressure of the chamber 10 to the thirdly set pressure. Thereafter, when the internal pressure of the chamber 10 reaches the thirdly set pressure, the control unit 50 may close the exhaust valve 32 so that the pressure is not reduced any more.

The reduction in pressure may remove cold air and moisture from the chamber 10, and allow the sterilant to be smoothly diffused when mixture gas including the sterilant is injected into the chamber 10.

For example, in the step of reducing the pressure of the chamber to the vacuum 310, the thirdly set pressure may be 300 to 1000 mTorr. The control unit 50 may control the vacuum unit 30 to reduce the internal pressure of the chamber 10 to a level that is close to almost 0 mTorr. When the internal pressure of the chamber 10 is more than 1000 mTorr, it may be difficult to sufficiently obtain a pressure reduction effect. When the internal pressure is less than 300 mTorr, the degree of vacuum may be unnecessarily reduced to increase a process cost.

In the sterilant injection step 320, the control unit 50 may control the sterilant supply unit 60 to supply the sterilant to the chamber 10. For example, the control unit 50 may perform controlling to open the sterilant supply valve 62 and operate the sterilant supply pump 61 to discharge the sterilant received in the sterilant supply tank 63 in a set amount.

Herein, the sterilant may include hydrogen peroxides. To be more specific, the sterilant may be a hydrogen peroxide solution having a concentration of 60% or less as designated by law.

The sterilant discharged from the sterilant supply tank 63 may be first injected into the vaporizer 20 before supplied to the chamber 10. The control unit 50 may control the vaporizer 20 to heat and vaporize the supplied sterilant. Subsequently, the control unit 50 may control the sterilant supply unit 60 and the air supply unit 40 to inject the sterilant into the chamber 10. Further, the heated sterilant may be injected in a vaporized form into the chamber 10.

In this case, the control unit 50 may control the sterilant supply unit 60 and the air supply unit 40 to continuously inject the vaporized sterilant into the chamber 10 so that the internal pressure of the chamber 10 becomes a fourthly set pressure that is lower than the atmospheric pressure. When the internal pressure of the chamber 10 reaches the fourthly set pressure, the control unit 50 may control the air supply unit 40 to maintain the internal pressure of the chamber 10 at the fourthly set pressure for a predetermined time.

For example, the fourthly set pressure may be 10 to 200 Torr. When the fourthly set pressure is more than 200 Torr, the amount of sterilant supplied into the chamber 10 may be increased to increase a process cost. When the fourthly set pressure is less than 10 Torr, the sterilant is not added in an appropriate amount, and thus the sterilant may be difficult to effectively permeate the sterilization target 80. In a reconsideration of the process cost and sterilization efficiency, the fourthly set pressure may be 20 to 100 Torr.

Subsequently, a sufficient time is provided to sufficiently diffuse the sterilant into the sterilization target 80. In this case, all the valves are in a closed state, and the power supply source 71 does not supply high frequency power. The time for which the fourthly set pressure is maintained may be two to ten times more than a time for which the pressure is increased to the fourthly set pressure. This is limited so that the sterilant is sufficiently diffused.

However, the time for which the fourthly set pressure is maintained is not limited to the aforementioned time as long as the sterilant is sufficiently diffused for the aforementioned time. However, the aforementioned time may be a process time that is relatively shorter than the process time of the sterilant diffusion step 330, and the process time will be described again later.

Subsequently, filtered external air may be supplied to the chamber 10. For example, the control unit 50 may control the air supply unit 40 to supply external air filtered by the HEPA filter 92 to the chamber 10. In the drawings, filtered external air is supplied through the vaporizer 20 to the chamber 10, but the present invention is not limited thereto. Accordingly, filtered external air is capable of being separately supplied while not passing through the vaporizer 20.

The control unit 50 may control the air supply unit 40 to supply filtered external air to the chamber 10 so that the internal pressure of the chamber 10 becomes the secondly set pressure that is higher than the atmospheric pressure. As described above, when the internal pressure of the chamber 10 is increased to the secondly set pressure that is higher than the atmospheric pressure, the sterilant may acceleratedly permeate the sterilization target 80.

Accordingly, the sterilant may permeate the inner cavity of the sterilization target 80 having the narrow and long inner cavity such as lumen.

For example, the secondly set pressure may be 900 to 760 Torr. When the secondly set pressure is more than 900 Torr, the internal pressure of the chamber 10 may be increased to reduce stability and lengthen an external air injection time to reduce productivity. When the secondly set pressure is less than 760 Torr, effective permeation of the sterilant may be insignificant.

In the sterilant diffusion step 330, the internal pressure of the chamber 10 is maintained at the secondly set pressure for a predetermined time. In this case, the sterilant may completely surround the sterilization target 80 when oxidation occurs to more deeply permeate the sterilization target 80. In this case, all the valves are in a closed state, and the power supply source 71 does not supply high frequency power.

For example, a ratio of the time of the sterilant diffusion step 330 to the time for which external air is injected may be two to ten times. In addition, the sterilant permeation step 330 may be performed for a longer time as compared to the time for which the fourthly set pressure is maintained. This is constituted to allow the sterilant to completely surround the sterilization target 80 at the secondly set pressure that is higher than the atmospheric pressure, thus more deeply permeating the sterilization target.

For example, a ratio of the time of the sterilant diffusion step 330 to the time for which the fourthly set pressure is maintained may be two to ten times. However, the process time of the sterilant diffusion step 330 is not limited to the aforementioned time as long as the sterilant is sufficiently diffused for the aforementioned process time.

In the sterilant discharging step 340, the internal pressure of the chamber 10 is reduced to the thirdly set pressure. For example, the control unit 50 may open the exhaust valve 32 and operate the vacuum pump 31 so that the internal pressure of the chamber 10 becomes the thirdly set pressure. In this case, when the internal pressure of the chamber 10 becomes the thirdly set pressure, the control unit 50 may control the exhaust valve 32 to close the exhaust valve 32.

In the plasma generation step 350, plasma is generated in the chamber 10. For example, the control unit 50 may operate the power supply source 71 to generate high frequency power so that plasma is generated in the chamber 10, and supply high frequency power to the electrode 72. When plasma is generated while the sterilant permeating the sterilization target 80 surrounds pathogenic bacteria, active oxygen and hydroxyl groups as free radicals are generated to kill even spores of microorganisms. Further, plasma converts the sterilant into a substance harmless to the environment.

For example, when hydrogen peroxides are used as the sterilant, plasma decomposes hydrogen peroxides into water and oxygen. In this case, in the present exemplary embodiment, plasma may be generated in the chamber 10 to simplify the apparatus. For reference, when plasma is generated in a separate vessel outside the chamber 10 and then supplied into the chamber 10, there is a problem in that the sterilization apparatus is complicated.

In the present invention, the sterilant injection step 320, the sterilant diffusion step 330, the sterilant discharging step 340, and the plasma generation step 350 may be repeatedly performed.

That is, in the plasma sterilization method, the sterilant injection step 321, the sterilant diffusion step 331, the sterilant discharging step 341, and the plasma generation step 351 may be additionally performed.

In the drawings and the detailed description, the sterilant injection step 320, the sterilant diffusion step 330, the sterilant discharging step 340, and the plasma generation step 350 are repeatedly performed two times, but the present invention is not limited thereto. Needless to say, the number of repetition may be adjusted in consideration of a type and a shape of the sterilization target 80 and a process condition during sterilization.

For example, the sterilant injection step 320, the sterilant diffusion step 330, the sterilant discharging step 340, and the plasma generation step 350 may be repeatedly performed two to five times. When the steps are repeatedly performed five times or more, a sterilization effect is not increased any more and only a process time and cost may be consumed. Further, even though the sterilant injection step 320, the sterilant diffusion step 330, the sterilant discharging step 340, and the plasma generation step 350 are performed once, there is no problem. However, when the steps are performed two times or more, the sterilization effect may be further improved.

The remaining gas removal step 370 may include a step of increasing the pressure of the chamber to the atmospheric pressure 371, and a remaining gas exhaust step 372.

In the step of increasing the pressure of the chamber to the atmospheric pressure 371, the internal pressure of the chamber 10 becomes the atmospheric pressure. For example, the control unit 50 may open the air supply valve for ventilation 91 until the internal pressure of the chamber 10 becomes the atmospheric pressure to supply external air through the HEPA filter 92 into the chamber 10.

In the remaining gas exhaust step 372, the control unit 50 may control the vacuum unit 30 to reduce the internal pressure of the chamber 10 to the fourthly set pressure that is lower than the atmospheric pressure. In this case, a small amount of harmful components (for example, hydrogen peroxide) may remain in the chamber 10. When air is exhausted from the chamber 10, a small amount of harmful components may be completely removed through the catalyst-type exhaust filter 33.

Subsequently, the control unit 50 may control the air supply valve for ventilation 91 to increase the internal pressure of the chamber 10 to the atmospheric pressure. The control unit 50 may open the air supply valve for ventilation 91 to supply external air filtered through the HEPA filter 92 into the chamber 10.

For example, the step of increasing the pressure of the chamber to the atmospheric pressure 371 and the remaining gas exhaust step 372 may be repeatedly performed for a set time.

Conditions, such as a time and a pressure, of a process of the remaining gas removal step 370 may be changed according to the shape of the chamber 10 and the sterilization target 80 or the sterilant.

Features, structures, and effects of the aforementioned content are included in at least one exemplary embodiment of the present invention, but are not limited to the one exemplary embodiment. Moreover, features, structures, and effects exemplified in each exemplary embodiment may be combined or modified by the person with ordinary skill in the art to which the exemplary embodiments belong with respect to other exemplary embodiments. Accordingly, contents relating to the combination and modification are to be construed to be included in the scope of the present invention.

What is claimed is:

1. A plasma sterilization apparatus comprising:
a chamber including a sterilization target;
a vaporizer heating air;
a vacuum unit discharging the air from the chamber to an outside;
an air supply unit injecting the heated air into the chamber; and
a control unit controlling the air supply unit to inject the air heated by the vaporizer into the chamber, controlling the vacuum unit to discharge the air to the outside until an internal pressure of the chamber becomes a firstly set pressure, and controlling the air supply unit to inject the heated air into the chamber until the internal pressure of the chamber becomes a secondly set pressure.

2. The plasma sterilization apparatus of claim 1, wherein the firstly set pressure has a value that is higher than 300 Torr and lower than an atmospheric pressure.

3. The plasma sterilization apparatus of claim 1, wherein the secondly set pressure has a value that is higher than an atmospheric pressure and lower than 900 Torr.

4. The plasma sterilization apparatus of claim 1, wherein the air supply unit includes
an air supply pump injecting the air heated by the vaporizer into the chamber; and
an injection valve provided between the chamber and the vaporizer and opened and closed to adjust an injection flow of the air heated by the vaporizer into the chamber, and
the control unit operates the air supply pump to inject the air heated by the vaporizer into the chamber and opens the injection valve to inject the air heated by the vaporizer into the chamber.

5. The plasma sterilization apparatus of claim 1, wherein the air supply unit includes
an air supply pump absorbing the air from the outside and supplying the filtered air to the vaporizer; and
a first air supply valve provided between the vaporizer and the air supply pump and opened and closed to adjust a supply flow of the filtered air to the vaporizer, and
the control unit operates the air supply pump to absorb the air from the outside and supply the filtered air to the vaporizer, and opens the first air supply valve to supply the filtered air to the vaporizer.

6. The plasma sterilization apparatus of claim 1, further comprising:
a sterilant supply unit supplying a sterilant to the chamber; and
a plasma generation unit generating plasma in the chamber.

7. A plasma sterilization method of a plasma sterilization apparatus, comprising:
heating air;
exhausting the air from a chamber to an outside until an internal pressure of the chamber becomes a firstly set pressure to perform a pressure reduction; and
injecting the heated air into the chamber until the internal pressure of the chamber becomes a secondly set pressure to perform a pressure increase.

8. The method of claim 7, wherein the exhausting of the air and the injecting of the heated air are repeatedly performed.

9. The method of claim 7, further comprising:
after the injecting of the heated air, reducing the pressure of the chamber to a set level, supplying a sterilant into the chamber, diffusing the sterilant for a set time, additionally reducing the pressure of the chamber, and generating plasma to perform sterilization.
